(19) United States
(12) Patent Application Publication
(10) Pub. No.: US 2026/0078124 A1
(43) Pub. Date: Mar. 19, 2026

(54) METHODS OF PREPARING ERGOLINE ANALOGS

(71) Applicant: THE REGENTS OF THE UNIVERSITY OF CALIFORNIA, Oakland, CA (US)

(72) Inventors: David E. OLSON, Oakland, CA (US); Jeremy R. TUCK, Oakland, CA (US)

(21) Appl. No.: 19/110,614

(22) PCT Filed: Sep. 11, 2023
(86) PCT No.: PCT/US2023/073837

§ 371 Date: Mar. 11, 2025

Related U.S. Application Data
(60) Provisional application No. 63/375,283, filed on Sep. 12, 2022.

(51) Int. Cl. C07D 471/16; C07D 213/82; C07D 401/04

(52) U.S. Cl. CPC C07D 471/16; C07D 213/82; C07D 401/04

(57) ABSTRACT

Provided herein are methods for preparing intermediate amide compounds for the preparation of ergoline analogs.

### CROSS-REFERENCE TO RELATED APPLICATIONS

This application claims priority to U.S. Provisional Application No. 63/375,283, filed Sep. 12, 2022, which is incorporated in its entirety herein for all purposes.

### STATEMENT AS TO RIGHTS TO INVENTIONS MADE UNDER FEDERALLY SPONSORED RESEARCH AND DEVELOPMENT

This invention was made with Government support under Grant No. GM128997, awarded by the National Institutes of Health. The Government has certain rights in the invention.

### BACKGROUND

Altered synaptic connectivity and plasticity has been observed in the brains of individuals with neuropsychiatric and neurological diseases/disorders. Psychoplastogens promote neuronal growth and improve neuronal architecture through mechanisms that may involve the activation of the serotonin 5-HT₂ receptors. Modulators of these biological targets, such as, for example, N,N-dimethyltryptamine (DMT), ibogaine, and lysergic acid diethylamide (LSD) have demonstrated psychoplastogenic properties. For example, LSD and other analogs of the ergoline scaffold are capable of rectifying deleterious changes in neuronal structure that are associated with neuropsychiatric and neurological diseases/disorders. Such structural alterations include, for example, the loss of dendritic spines and synapses in the prefrontal cortex (PFC) as well as reductions in dendritic arbor complexity. Furthermore, pyramidal neurons in the PFC exhibit top-down control over areas of the brain controlling motivation, fear, reward, and cognition. Hallucinogenic psychoplastogens have demonstrated antidepressant, anxiolytic, and anti-addictive effects in the clinic. However, their subjective effects have limited their clinical utility. Moreover, hallucinogenic compounds are contraindicated for psychotic illnesses like schizophrenia, which are well known to involve the loss of dendritic spines in the PFC. Thus, non-hallucinogenic psychoplastogens may have distinct advantages over their hallucinogenic counterparts.

Provided herein are methods of preparing compounds with clinically relevant therapeutic efficacy that have improved physicochemical properties.

### BRIEF SUMMARY OF THE INVENTION

In one embodiment, the present invention provides a method of preparing a compound of Formula I:

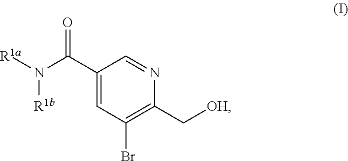

the method comprising:
(a1) forming a first reaction mixture comprising a first activating agent, and a compound of Formula II:

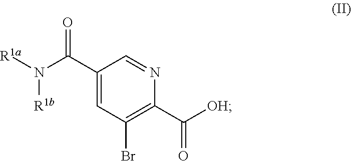

and
(a2) adding the first reaction mixture to a solution comprising a reducing agent, under conditions suitable to prepare the compound of Formula I,
   wherein R^{1a} and R^{1b} are each independently C₁₋₆ alkyl, C₁₋₆ alkoxy, C₁₋₆ alkoxyalkyl, C₁₋₆ haloalkyl, C₁₋₆ haloalkoxy, or C₃₋₆ cycloalkyl; alternatively, R^{1a} and R^{1b} are combined to form a 4 to 8 membered heterocycloalkyl having 1 to 2 heteroatoms, each independently N, O, or S.

In another embodiment, the present invention provides a method of preparing a compound of Formula IX:

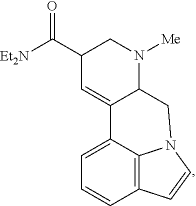

the method comprising:
(d1) forming a fourth reaction mixture comprising oxalyl chloride, and a compound of Formula V:

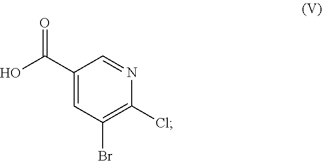 [SMILES: O=C(O)c1cnc(Cl)c(Br)c1]

(d2) adding diethylamine to the fourth reaction mixture, under conditions suitable to prepare a compound of Formula IV:

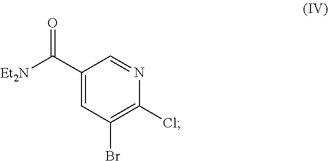

(c) forming a third reaction mixture comprising ethylcyanoacetate, potassium carbonate, dimethylformamide, and the compound of Formula IV, under conditions suitable to prepare a compound of Formula III:

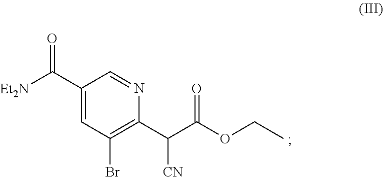

(b1) forming a second reaction mixture comprising sodium hydroxide, water, and the compound of Formula III;
(b2) adding hydrogen peroxide to the second reaction mixture, under conditions suitable to prepare a compound of Formula II having the structure:

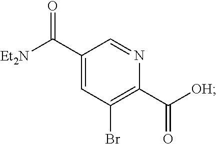

(a1) forming a first reaction mixture comprising carbonyldiimidazole and the compound of Formula II;
(a2) adding the first reaction mixture to a solution comprising sodium borohydride, under conditions suitable to prepare a compound of Formula I having the structure:

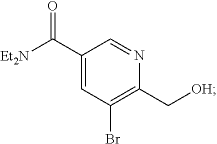

(e1) forming a fifth reaction mixture comprising methyliodide, and the compound of Formula I, under conditions suitable to prepare a methyl pyridinium salt of Formula VI:

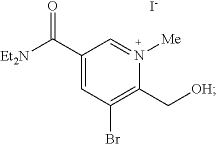

(e2) forming a sixth reaction mixture comprising sodium cyanoborohydride, and the methyl pyridinium salt of Formula VI, under conditions suitable to prepare a compound of Formula VII:

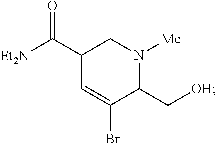

(f) forming a seventh reaction mixture comprising indole-7-boronic acid pinacol ester, sodium carbonate, Pd(PPh₃)₄, and the compound of Formula VII, under conditions suitable to prepare a compound of Formula VIII:

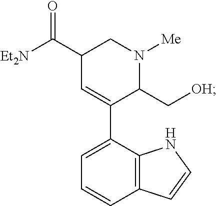

and
(g) forming an eighth reaction mixture comprising tosyl-chloride, sodium hydroxide, and the compound of Formula VIII, under conditions suitable to prepare the compound of Formula IX.

### DETAILED DESCRIPTION OF THE INVENTION

### I. General

Provided herein are methods for preparing tetracyclic ergoline analogs of heterocyclic compounds.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Forming a reaction mixture” refers to the process of bringing into contact at least two distinct species such that they mix together and can react. It should be appreciated, however, the resulting reaction product can be produced directly from a reaction between the added reagents or from an intermediate from one or more of the added reagents which can be produced in the reaction mixture.

“Activating agent” refers to a reagent capable of forming an activated ester from a carboxylic acid to faciliate reaction at the carbonyl carbon, such as addition of hydrogen atoms to reduce the carbonyl to a methylene, or formation of a carbon-carbon or carbon-nitrogen bond at the carbonyl carbon. Representative carboxyl coupling agent include thionyl chloride, carbodiimide reagents, oxalyl chloride, and carbonyldiimidazole, and others.

“Reducing agent” refers to a reagent capable of donating an electron to another compound, thus reducing the compound. Representative reducing agents include, but are not limited to, hydrogenation catalysts, hydride reagents such as lithium aluminum hydride (LiAlH₄) or sodium borohydride (NaBH₄), sodium, formic acid, etc. Hydrogenation catalysts are catalysts that facilitate the hydrogenation of an alkene or other double bond. Representative hydrogenation catalysts include, but are not limited to, Pd/C, Pt, Raney nickel, Ru complexes, Ir complexes, etc.

“Base” refers to a compound capable of accepting a proton (a Bronsted-Lowry base) or capable of donating an electron pair (a Lewis base). Representative bases include, but are not limited to, inorganic bases, organic bases, acid salts, non-nucleophlic bases, and amine bases. For example, the base can be sodium hydroxide, sodium acetate, or mixtures thereof. “Amine base” or “non-nucleophilic amine base” refers to a nitrogen-containing base that is a moderate to strong base but at the same time is a poor nucleophile. Representative amine bases include bases such as triethylamine, diisopropylethyl amine, N,N-diethylaniline, pyridine, 2,6-lutidine, 2,4,6-collidine, 4-dimethylaminopyridine, and quinuclidine.

“Inorganic base” refers to hydroxides, carbonates, and acetates, having a metal counterion. “Metal” refers to elements of the periodic table that are metallic and that can be neutral, or negatively or positively charged as a result of having more or fewer electrons in the valence shell than is present for the neutral metallic element. Metals useful in the present invention include the alkali metals, alkali earth metals, transition metals and post-transition metals. Alkali metals include Li, Na, K, Rb and Cs. Alkaline earth metals include Be, Mg, Ca, Sr and Ba. Transition metals include Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg and Ac. Post-transition metals include Al, Ga, In, Ti, Ge, Sn, Pb, Sb, Bi, and Po. Rare earth metals include Sc, Y, La, Ce, Pr, Nd, Sn, Eu, Gd, Tb, Dy, Ho, Er, Tm, Yb and Lu. One of skill in the art will appreciate that the metals described above can each adopt several different oxidation states, all of which are useful in the present invention. In some instances, the most stable oxidation state is formed, but other oxidation states are useful in the present invention.

“Oxidizing agent” or “oxidizer” refers to a reagent capable of accepting an electron pair from another compound, thus oxidizing the compound. Representative oxidizing agents include, but are not limited to, oxygen, hydrogen peroxide, nitrite, nitric acid, sulfuric acids, etc.

“Alkyl” refers to a straight or branched, saturated, aliphatic radical having the number of carbon atoms indicated. Disclosures provided herein of an “alkyl” are intended to include independent recitations of a saturated alkyl, unless otherwise stated. Alkyl groups described herein are generally monovalent, but may also be divalent which may also be described herein as “alkylene” or “alkylenyl” groups. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₄, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₀₋₆, C₁₋₂, C₁₋₃, C₁₋₄, C₁₋₃, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The alkoxy component is as defined above. Examples of the alkoxyalkyl group include, but are not limited to, 2-ethoxy-ethyl and methoxymethyl.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. In some embodiments, cycloalkyls are spirocyclic or bridged compounds. In some embodiments, cycloalkyls are optionally fused with an aromatic ring, and the point of attachment is at a carbon that is not an aromatic ring carbon atom. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. Cycloalkyl groups can contain one or more double bonds in the ring.

“Heterocycloalkyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

### III. Methods of Making

In some embodiments, the present invention provides a method of preparing a compound of Formula I:

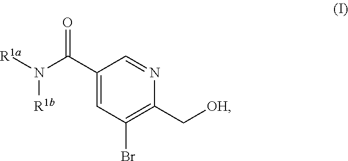

the method comprising:
(a1) forming a first reaction mixture comprising a first activating agent, and a compound of Formula II:

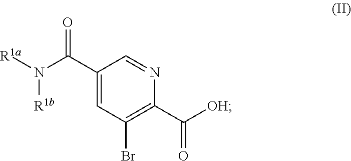

and
(a2) adding the first reaction mixture to a solution comprising a reducing agent, under conditions suitable to prepare the compound of Formula I,
   wherein R^{1a} and Rib are each independently C₁₋₆ alkyl, C₁₋₆ alkoxy, C₁₋₆ alkoxyalkyl, C₁₋₆ haloalkyl, C₁₄ haloalkoxy, or C₃₋₆ cycloalkyl; alternatively, R^{1a} and R^{1b} are combined to form a 4 to 8 membered heterocycloalkyl having 1 to 2 heteroatoms, each independently N, O, or S.

In some embodiments, the method of the present invention is the method wherein R^{1a} and R^{1b} are each independently C₁₋₆ alkyl. In some embodiments, the method of the present invention is the method wherein R^{1a} and R^{1b} are each independently methyl, ethyl, n-propyl, or i-propyl. In some embodiments, the method of the present invention is the method wherein R^{1a} and Rib are each ethyl.

In some embodiments, the method of the present invention is the method wherein the compound of Formula I is a compound of Formula Ia:

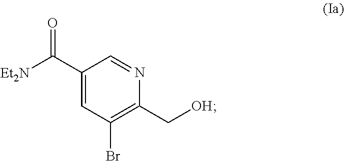

and
the compound of Formula II is a compound of Formula IIa:

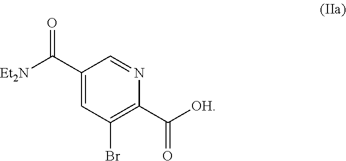

In some embodiments, the present invention provides a method of preparing a compound of Formula Ia having the structure:

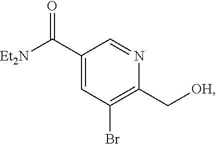

the method comprising:
(a1) forming a first reaction mixture comprising a first activating agent, and a compound of Formula Ha having the structure:

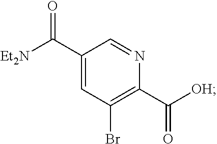

and
(a2) adding the first reaction mixture to a solution comprising a reducing agent, under conditions suitable to prepare the compound of Formula Ia.

The first activating agent of the method of the present invention can be any agent capable of activating a carboxylic acid group for reduction to the hydroxymethyl group. For example, the first activating agent includes, but is not limited to, a carbodiimide, thionyl chloride, oxalyl chloride, a uronium, and carbonyldiimidazole.

In some embodiments, the method of the present invention is the method wherein the first activating agent comprises carbonyldiimidazole, dicyclohexylcarbodiimide (DCC), N,N′-diisopropylcarbodiimide (DIC), 1-ethyl-3-(3-dimethylaminopropyl)carbodiimide (EDC), 1-ethyl-3-(3-dimethylaminopropyl)carbodiimide HCl (EDAC), bis[[4-(2,2-dimethyl-1,3-dioxolyl)]methyl]carbodiimide (BDDC), N-cyclohexyl-N′-(2-morpholinoethyl)carbodiimide methyl-p-toluenesulfonate, 1-[3-(dimethylamino)propyl]-3-ethylcarbodiimide methiodide, thionyl chloride, oxalyl chloride, 2-ethoxy-1-ethoxycarbonyl-1,2-dihydroquinoline (EEDQ), carbonyl diimidazole, bis(1,2,4-triazolyl)methanone, n-propanephosphonic acid anhydride, ethylmethylphosphonic anhydride (EMPA), cyanuric chloride, 2-chloro-4,6-dimethoxy-1,3,5-triazine (CDMT), 4-(4,6-dimethoxy-1,3,5-triazin-2-yl)-4-methylmorphonium chloride (DMTMM), guanidinium salt, uronium salt, hexafluorophosphate benzotriazole tetramethyl uronium (HBTU), hexafluorophosphate azabenzotriazole tetramethyl uronium (HATU), 2-(1H-Benzotriazole-1-yl)-1,1,3,3-tetramethylaminium tetrafluoroborate (TBTU), O-(2-Oxo-1(2H)pyridyl)-N,N,N′,N′-tetramethyluronium tetrafluoroborate (TPTU), or N-[[[(1-Cyano-2-ethoxy-2-oxoethylidene)amino]oxy](dimethylamino)methylene]-N-methyl-methanaminium tetrafluoroborate (TOTU). In some embodiments, the method of the present invention is the method wherein the first activating agent comprises carbonyldiimidazole.

The first activating agent can be present in the first reaction mixture in any suitable amount. For example, the first activating agent can be present in the first reaction mixture in an amount of from 0.1 to 10 molar equivalents to the compound of Formula II or IIa, or 0.5 to 5 molar equivalents, or from 1 to 5 molar equivalents, or from 1 to 3 molar equivalents to the compound of Formula II or IIa. Representative amounts of the first activating agent in the first reaction mixture include, but are not limited to, about 1.0 molar equivalents to the compound of Formula II or IIa, or 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 molar equivalents to the compound of Formula II or IIa.

The reducing agent can be any reducing agent suitable for reducing a carboxylic acid to a hydroxymethyl group. In some embodiments, the reducing agent comprises a hydrogenation catalyst, triethylsilane/iron(III) chloride hexahydrate (Et₃SiH/FeCl₃·6H₂O), sodium borohydride in pyridine [(NaBH₄)/pyridine], tri-n-butyltin hydride [(nBu₃SnH)], tri-(trimethysilyl)silane [(Me₃Si)₃SiH], borane (BH₃), and catechol borane [(o-C₆H₄O₂)BH], hydrazine (H₂NNH₂), formic acid, ammonium formate, cyclohexene, or 1,4-cyclohexadiene. When the reducing agent is the hydrogenation catalyst, the first reaction mixture further comprises hydrogen.

In some embodiments, the method of the present invention is the method wherein the reducing agent comprises lithium borohydride (LiBH₄), sodium borohydride (NaBH₄), calcium borohydride (Ca(BH₄)₂), sodium cyanoborohydride (NaBH₃CN), sodium triacetoxyborohydride (NaBH(OAc)₃), diisobutylaluminum hydride (DIBAL), or lithium aluminum hydride (LiAlH₄). In some embodiments, the method of the present invention is the method wherein the reducing agent comprises sodium borohydride.

The reducing agent can be present in the first reaction mixture in any suitable amount. For example, the reducing agent can be present in the first reaction mixture in an amount of from 0.1 to 10 molar equivalents to the compound of Formula II or Ha, or 0.5 to 5 molar equivalents, or from 1 to 5 molar equivalents, or from 2 to 4 molar equivalents to the compound of Formula II or IIa. Representative amounts of the reducing agent in the first reaction mixture include, but are not limited to, about 2.0 molar equivalents to the compound of Formula II or IIa, or 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or about 4.0 molar equivalents to the compound of Formula II or Ha.

In some embodiments, the method of the present invention is the method wherein the first activating agent is present in an amount of 1 to 5 molar equivalents to the compound of Formula II; and the reducing agent is present in an amount of 1 to 5 molar equivalents to the compound of Formula II. In some embodiments, the method of the present invention is the method wherein the first activating agent is present in an amount of 1 to 3 molar equivalents to the compound of Formula II; and the reducing agent is present in an amount of 2 to 4 molar equivalents to the compound of Formula II.

The first reaction mixture can be performed at any suitable temperature. Suitable temperatures for the first reaction mixture can be from −50° C. to 100° C., or from −25° C. to 75° C., or from 0° C. to 60° C. In some embodiments, the method of the present invention is the method wherein the temperature of the first reaction mixture is from 0° C. to 60° C.

The first reaction mixture can be at any suitable pressure. For example, the first reaction mixture can be at atmospheric pressure. The first reaction mixture can also be under an inert atmosphere.

In some embodiments, the method of the present invention is the method wherein the compound of Formula II is prepared by the method comprising: (a1) forming the first reaction mixture comprising carbonyldiimidazole and the compound of Formula II; and (a2) adding the first reaction mixture to the solution comprising sodium borohydride, under conditions suitable to prepare the compound of Formula I.

In some embodiments, the method of the present invention is the method wherein the compound of Formula II is prepared by the method comprising:
(b1) forming a second reaction mixture comprising a first inorganic base, water, and a compound of Formula III:

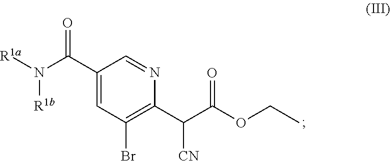

and
(b2) adding an oxidizing agent to the second reaction mixture, under conditions suitable to prepare the compound of Formula II.

In some embodiments, the method of the present invention is the method wherein the compound of Formula Ha is prepared by the method comprising:
(b1) forming a second reaction mixture comprising a first inorganic base, water, and a compound of Formula IIIa:

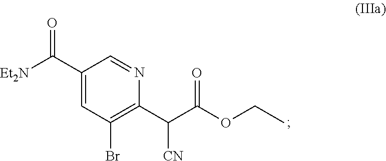

and
(b2) adding an oxidizing agent to the second reaction mixture, under conditions suitable to prepare the compound of Formula IIa.

The first inorganic base of the second reaction mixture can be any suitable first inorganic base. For example, the first inorganic base can be any metal hydroxide, carbonate or acetate such as, but not limited to, lithium hydroxide, sodium hydroxide, potassium hydroxide, sodium carbonate, potassium carbonate, cesium carbonate, or sodium acetate

In some embodiments, the method of the present invention is the method wherein the first inorganic base comprises lithium hydroxide, sodium hydroxide, potassium hydroxide, sodium carbonate, potassium carbonate, cesium carbonate, or sodium acetate. In some embodiments, the method of the present invention is the method wherein the first inorganic base comprises sodium hydroxide.

The first inorganic base can be present in the second reaction mixture in any suitable amount. For example, the first inorganic base can be present in the second reaction mixture in an amount of from 0.1 to 10 molar equivalents to the compound of Formula III or IIIa, or 0.5 to 5 molar equivalents, or from 1 to 5 molar equivalents, or from 1.5 to 3.5 molar equivalents to the compound of Formula III or IIIa. Representative amounts of the first inorganic base in the second reaction mixture include, but are not limited to, about 1.5 molar equivalents to the compound of Formula III or IIIa, or 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or about 3.5 molar equivalents to the compound of Formula III or IIIa.

The second reaction mixture can include any suitable oxidizing agent capable of oxidizing a cyanoalkylester to a carboxylic acid. Representative oxidizing agents useful in the second reaction mixture include, but are not limited to, any alkyl hydroperoxides, any peroxyacid, any peracid, and others.

In some embodiments, the method of the present invention is the method wherein the oxidizing agent comprises hydrogen peroxide, tert-butylhydroperoxide, meta-chloroperoxybenzoic acid (mCPBA), peracetic acid, or oxone. In some embodiments, the method of the present invention is the method wherein the oxidizing agent comprises hydrogen peroxide.

The oxidizing agent can be present in the second reaction mixture in any suitable amount. For example, the oxidizing agent can be present in the second reaction mixture in an amount of from 1 to 20 molar equivalents to the compound of Formula III or IIIa, or 1 to 15 molar equivalents, or from 2 to 10 molar equivalents, or from 4 to 8 molar equivalents to the compound of Formula III or IIIa. Representative amounts of the oxidizing agent in the second reaction mixture include, but are not limited to, about 4.0 molar equivalents to the compound of Formula III or IIIa, or 4.5, 5.0, 5.5, 6.0, 6.5, 7.0, 7.5, or about 8.0 molar equivalents to the compound of Formula III or IIIa.

In some embodiments, the method of the present invention is the method wherein the first inorganic base is present in an amount of 1.5 to 3.5 molar equivalents to the compound of Formula IIIa; and the oxidizing agent is present in an amount of 4 to 8 molar equivalents to the compound of Formula IIIa.

The second reaction mixture can be performed at any suitable temperature. Suitable temperatures for the second reaction mixture can be from −50° C. to 150° C., or from −50° C. to 100° C., or from −30° C. to 100° C. In some embodiments, the method of the present invention is the method wherein the temperature of the second reaction mixture is from −30° C. to 100° C.

The second reaction mixture can be at any suitable pressure. For example, the second reaction mixture can be at atmospheric pressure. The second reaction mixture can also be under an inert atmosphere.

In some embodiments, the method of the present invention is the method wherein the compound of Formula II is prepared by the method comprising:
(b1) forming the second reaction mixture comprising sodium hydroxide, water, and the compound of Formula IIIa:

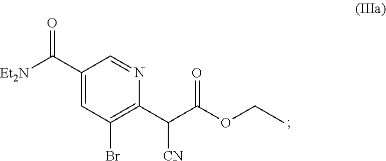

and
(b2) adding hydrogen peroxide to the second reaction mixture, under conditions suitable to prepare the compound of Formula II.

In some embodiments, the method of the present invention is the method wherein the compound of Formula III is prepared by the method comprising:
(c) forming a third reaction mixture comprising ethylcyanoacetate, a second inorganic base, and a compound of Formula IV:

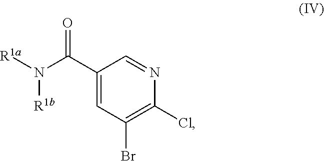

under conditions suitable to prepare the compound of Formula III.

In some embodiments, the method of the present invention is the method wherein the compound of Formula IIIa is prepared by the method comprising:
(c) forming a third reaction mixture comprising ethylcyanoacetate, a second inorganic base, and a compound of Formula IVa:

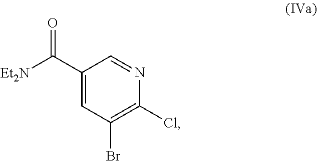

under conditions suitable to prepare the compound of Formula IIIa.

The ethylcyanoacetate can be present in the third reaction mixture in any suitable amount. For example, the ethylcyanoacetate can be present in the third reaction mixture in an amount of from 0.1 to 10 molar equivalents to the compound of Formula IV or IVa, or 1 to 10 molar equivalents, or from 1 to 5 molar equivalents, or from 2 to 5 molar equivalents to the compound of Formula IV or IVa. Representative amounts of the ethylcyanoacetate in the third reaction mixture include, but are not limited to, about 2.0 molar equivalents to the compound of Formula IV or IVa, or 2.5, 3.0, 3.5, 4.0, 4.5, or about 5.0 molar equivalents to the compound of Formula IV or IVa.

The third reaction mixture includes any suitable second inorganic base. For example, the second inorganic base can include, but is not limited to, sodium carbonate, potassium carbonate, cesium carbonate, sodium hydride, lithium hexamethyldisiloxane (LiHMDS), potassium hexamethyldisiloxane (KHMDS), sodium hexamethyldisiloxane (NaHMDS), and lithium diisopropyl amine (LDA)

In some embodiments, the method of the present invention is the method wherein the second inorganic base comprises sodium carbonate, potassium carbonate, cesium carbonate, sodium hydride, lithium hexamethyldisiloxane (LiHMDS), potassium hexamethyldisiloxane (KHMDS), sodium hexamethyldisiloxane (NaHMDS), or lithium diisopropyl amine (LDA). In some embodiments, the method of the present invention is the method wherein the second inorganic base comprises potassium carbonate.

The second inorganic base can be present in the third reaction mixture in any suitable amount. For example, the second inorganic base can be present in the third reaction mixture in an amount of from 0.1 to 20 molar equivalents to the compound of Formula IV or IVa, or 1 to 20 molar equivalents, or from 2 to 12 molar equivalents, or from 4 to 10 molar equivalents to the compound of Formula IV or IVa. Representative amounts of the second inorganic base in the third reaction mixture include, but are not limited to, about 4.0 molar equivalents to the compound of Formula IV or IVa, or 4.5, 5.0, 5.5, 6.0, 6.5, 7.0, 7.5, 8.0, 8.5, 9.0, 9.5, or about 10.0 molar equivalents to the compound of Formula IV or IVa.

In some embodiments, the method of the present invention is the method wherein the ethylcyanoacetate is present in an amount of 2.0 to 5.0 molar equivalents to the compound of Formula IVa; and the second inorganic base is present in an amount of 4 to 10 molar equivalents to the compound of Formula IVa.

The third reaction mixture can include additional components. In some embodiments, the method of the present invention is the method wherein the third reaction mixture further comprises dimethylformamide.

The third reaction mixture can be at any suitable temperature. Suitable temperatures for the third reaction mixture can be from 25° C. to 200° C., or from 50° C. to 200° C., or from 80° C. to 150° C. In some embodiments, the method of the present invention is the method wherein the temperature of the third reaction mixture is from 80° C. to 150° C.

The third reaction mixture can be at any suitable pressure. For example, the third reaction mixture can be at atmospheric pressure. The third reaction mixture can also be under an inert atmosphere.

In some embodiments, the method of the present invention is the method wherein the compound of Formula IVa is prepared by the method comprising: (c) forming the third reaction mixture comprising ethylcyanoacetate, potassium carbonate, dimethylformamide, and the compound of Formula IVa, under conditions suitable to prepare the compound of Formula IIIa.

In some embodiments, the method of the present invention is the method wherein the compound of Formula IV is prepared by the method comprising:
(d1) forming a fourth reaction mixture comprising a second activating agent, and a compound of Formula V:

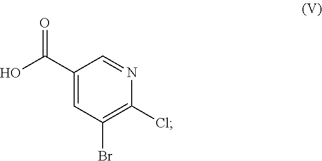 [SMILES: O=C(O)c1cnc(Cl)c(Br)c1]

and
(d2) adding (Ra^{1a})(R^{1b})NH to the fourth reaction mixture, under conditions suitable to prepare the compound of Formula IV.

In some embodiments, the method of the present invention is the method wherein the compound of Formula IVa is prepared by the method comprising:
(d1) forming a fourth reaction mixture comprising a second activating agent, and a compound of Formula V:

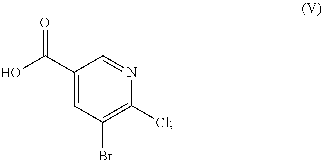 [SMILES: O=C(O)c1cnc(Cl)c(Br)c1]

and
(d2) adding diethylamine to the fourth reaction mixture, under conditions suitable to prepare the compound of Formula IVa.

The second activating agent of the method of the present invention can be any agent capable of activating a carboxylic acid group for reduction to the hydroxymethyl group. For example, the second activating agent includes, but is not limited to, a carbodiimide, thionyl chloride, oxalyl chloride, a uronium, and carbonyldiimidazole.

In some embodiments, the method of the present invention is the method wherein the second activating agent comprises oxalyl chloride, thionyl chloride, phosphorous oxychloride, (chloromethylene)dimethyliminium chloride, phosphorous(V) chloride, phosphorous(III) chloride, carbonyldiimidazole, dicyclohexylcarbodiimide (DCC), N,N′-diisopropylcarbodiimide (DIC), 1-ethyl-3-(3-dimethylaminopropyl)carbodiimide (EDC), 1-ethyl-3-(3-dimethylaminopropyl)carbodiimide HCl (EDAC), bis[[4-(2,2-dimethyl-1,3-dioxolyl)]methyl]carbodiimide (BDDC), N-cyclohexyl-N′-(2-morpholinoethyl)carbodiimide methyl-p-toluenesulfonate, 1-[3-(dimethylamino)propyl]-3-ethylcarbodiimide methiodide, thionyl chloride, oxalyl chloride, 2-ethoxy-1-ethoxycarbonyl-1,2-dihydroquinoline (EEDQ), carbonyl diimidazole, bis(1,2,4-triazolyl)methanone, n-propanephosphonic acid anhydride, ethylmethylphosphonic anhydride (EMPA), cyanuric chloride, 2-chloro-4,6-dimethoxy-1,3,5-triazine (CDMT), 4-(4,6-dimethoxy-1,3,5-triazin-2-yl)-4-methylmorphonium chloride (DMTMM), guanidinium salt, uronium salt, hexafluorophosphate benzotriazole tetramethyl uronium (HBTU), hexafluorophosphate azabenzotriazole tetramethyl uronium (HATU), 2-(1H-Benzotriazole-1-yl)-1,1,3,3-tetramethylaminium tetrafluoroborate (TBTU), 0-(2-Oxo-1(2H)pyridyl)-N,N,N′,N′-tetramethyluronium tetrafluoroborate (TPTU), or N-[[[(1-Cyano-2-ethoxy-2-oxoethylidene)amino]oxy](dimethylamino)methylene]-N-methyl-methanaminium tetrafluoroborate (TOTU). In some embodiments, the method of the present invention is the method wherein the second activating agent comprises oxalyl chloride.

The second activating agent can be present in the fourth reaction mixture in any suitable amount. For example, the second activating agent can be present in the fourth reaction mixture in an amount of from 0.1 to 10 molar equivalents to the compound of Formula V, or 0.5 to 5 molar equivalents, or from 1 to 5 molar equivalents, or from 1 to 3 molar equivalents to the compound of Formula V. Representative amounts of the second activating agent in the fourth reaction mixture include, but are not limited to, about 1.0 molar equivalents to the compound of Formula V, or 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or about 3.0 molar equivalents to the compound of Formula V.

The amine can be present in the fourth reaction mixture in any suitable amount. For example, the amine can be present in the fourth reaction mixture in an amount of from 0.1 to 10 molar equivalents to the compound of Formula V, or 1 to 10 molar equivalents, or from 1 to 8 molar equivalents, or from 3 to 6 molar equivalents to the compound of Formula V. Representative amounts of the amine in the fourth reaction mixture include, but are not limited to, about 3.0 molar equivalents to the compound of Formula V, or 3.5, 4.0, 4.5, 5.0, 5.5, or about 6.0 molar equivalents to the compound of Formula V.

In some embodiments, the method of the present invention is the method wherein the second activating agent is present in an amount of 1 to 3 molar equivalents to the compound of Formula V; and the diethylamine is present in an amount of 3 to 6 molar equivalents to the compound of Formula V.

The fourth reaction mixture can be performed at any suitable temperature. Suitable temperatures for the fourth reaction mixture can be from −50° C. to 100° C., or from −25° C. to 75° C., or from −10° C. to 40° C. In some embodiments, the method of the present invention is the method wherein the temperature of the fourth reaction mixture is from −10° C. to 40° C.

The fourth reaction mixture can be at any suitable pressure. For example, the fourth reaction mixture can be at atmospheric pressure. The fourth reaction mixture can also be under an inert atmosphere.

In some embodiments, the method of the present invention is the method wherein (d1) forming the fourth reaction mixture comprising oxalyl chloride, and the compound of Formula V; and (d2) adding diethylamine to the fourth reaction mixture, under conditions suitable to prepare the compound of Formula IVa.

In some embodiments, the present invention provides a method of preparing a compound of Formula IX:

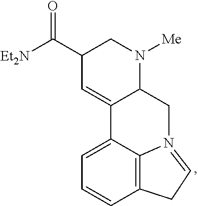

the method comprising:
(d1) forming a fourth reaction mixture comprising oxalyl chloride, and a compound of Formula V:

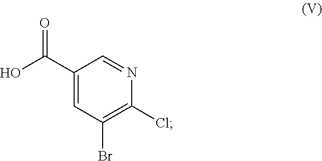 [SMILES: O=C(O)c1cnc(Cl)c(Br)c1]

(d2) adding diethylamine to the fourth reaction mixture, under conditions suitable to prepare a compound of Formula IVa:

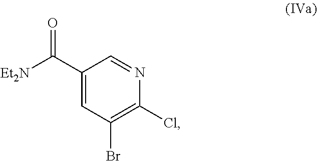

(c) forming a third reaction mixture comprising ethylcyanoacetate, potassium carbonate, dimethylformamide, and the compound of Formula IVa, under conditions suitable to prepare a compound of Formula IIIa:

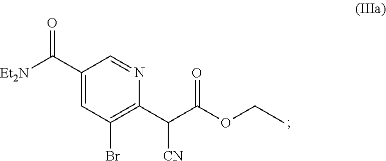

(b1) forming a second reaction mixture comprising sodium hydroxide, water, and the compound of Formula IIIa;
(b2) adding hydrogen peroxide to the second reaction mixture, under conditions suitable to prepare a compound of Formula IIa:

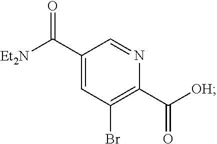

(a1) forming a first reaction mixture comprising carbonyldiimidazole and the compound of Formula Ia;
(a2) adding the first reaction mixture to a solution comprising sodium borohydride, under conditions suitable to prepare a compound of Formula Ia:

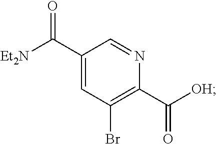

(e1) forming a fifth reaction mixture comprising methyliodide, and the compound of Formula Ia, under conditions suitable to prepare a methyl pyridinium salt of Formula VI:

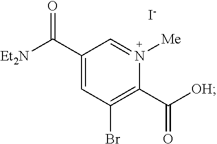

(e2) forming a sixth reaction mixture comprising sodium cyanoborohydride, and the methyl pyridinium salt of Formula VI, under conditions suitable to prepare a compound of Formula VII:

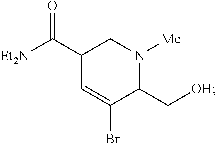

(f) forming a seventh reaction mixture comprising indole-7-boronic acid pinacol ester, sodium carbonate, Pd(PPh₃)₄, and the compound of Formula VII, under conditions suitable to prepare a compound of Formula VIII:

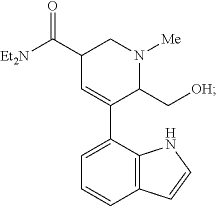

and
(g) forming an eighth reaction mixture comprising tosyl-chloride, sodium hydroxide, and the compound of Formula VIII, under conditions suitable to prepare the compound of Formula IX.

In some embodiments, the compound of Formula VIII has the structure:

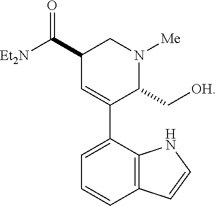

In some embodiments, the compound of Formula VIII has the structure:

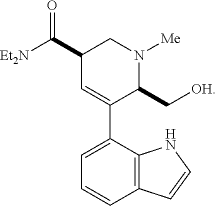

In some embodiments, the compound of Formula IX has the structure:

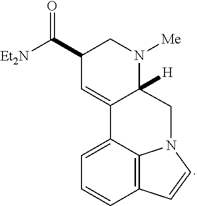

In some embodiments, the compound of Formula IX has the structure:

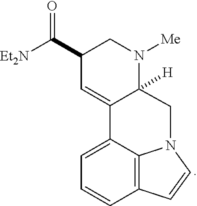

### IV. Examples

All reagents and solvents were obtained from commercial sources and used as received. Reactions were performed using oven-dried glassware (120° C.) under an inert N₂ atmosphere unless otherwise noted. Air- and moisture-sensitive liquids and solutions were transferred via syringe or stainless-steel cannula. Organic solutions were concentrated under reduced pressure (˜5 Torr) by rotary evaporation.

Nuclear magnetic resonance (NMR) spectra were acquired on a Bruker 400 operating at 400 and 100 MHz for ¹H and ¹³C, respectively, and are referenced internally according to residual solvent signals. Data for ¹H NMR are recorded as follows: chemical shift (8, ppm), multiplicity (s, singlet; d, doublet; t, triplet; q, quartet; quint, quintet; m, multiplet), coupling constant (Hz), and integration. Data for ¹³C NMR are reported in terms of chemical shift (8, ppm). Liquid chromatography-mass spectrometry (LC-MS) was performed using a Waters Alliance 2695 HPLC with a Waters Micromass ZQ Detector or Waters LC-MS with an ACQUITY Arc QDa detector.

### Example 1: Preparation of 5-bromo-N,N-diethyl-6-(hydroxymethyl)nicotinamide

### 5-bromo-6-chloro-N,N-diethylnicotinamide

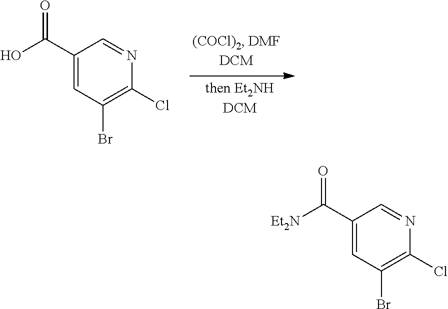

To a 0° C. cooled solution of 5-bromo-6-chloronicotinic acid (25.012 g, 105.781 mmol, 1.0 equiv), DCM (265 mL), and DMF (1 mL) was added oxalyl chloride (22.68 mL, 264.453 mmol, 2.5 equiv) dropwise over 20 minutes. The mixture was warmed to ambient temperature and stirring was continued for 2.5 h before removing the volatiles under reduced pressure. The residue was taken up in DCM (530 mL), cooled to 0° C., and a solution of diethylamine (43.8 mL, 423.124 mmol, 4.0 equiv) in DCM (265 mL) was added slowly over 30 minutes via cannula. The solution was warmed to ambient temperature and then concentrated to ˜% volume. Next, H₂O (1000 mL) was added followed by the slow addition of 4M HCl_{(aq)} (60 mL) with vigorous stirring. The layers were separated, and the aqueous layer was further extracted with DCM (3×150 mL). The combined organic layers were washed with saturated NaHCO_{3(aq)} (2×200 mL) and then with brine (2×200 mL). The organic solution was stirred with activated charcoal (15 g) and then filtered over celite. The filter cake was washed with additional DCM (400 mL) and the pale-yellow organic solution was concentrated under reduced pressure. The residue was dissolved in Et₂O (20.0 mL) and seeded with 0.010 g of pure compound (obtained previously by silica gel chromatography using 25% EtOAC in Hexanes) and left to sit at room temperature for 1 hour. Once a sizeable crystal bed was formed, hexanes (17.5 mL) were added with gentle swirling, then the mixture was cooled to 0° C. for 1 h and subsequently in a −20° C. freezer for 4 hours. The solvent was decanted and the solid was washed with ice-cold hexanes (2×25 mL) then dried in vacuo to obtain 5-bromo-6-chloro-N,N-diethylnicotinamide (26.998 g, 88%) as a white crystalline solid.

¹H NMR (400 MHz, CDCl₃) δ=8.34 (d, J=2.04 Hz, 1H), 7.96 (d, J=2.04 Hz, 1H), 3.59-3.42 (m, 2H), 3.36-3.18 (m, 2H), 1.28-1.10 (m, 6H) ppm. ¹³C NMR (100 MHz, CDCl₃) 5=166.0, 151.5, 145.3, 140.7, 133.0, 120.5, 43.6, 39.9, 14.4, 12.9 ppm. LRMS (ES⁺) m/z [M+H]⁺ calcd for C₁₀H₁₃BrClN₂O⁺ 290.99; Found 291.09.

### Ethyl 2-(3-bromo-5-(diethylcarbamoyl)pyridin-2-yl)-2-cyanoacetate

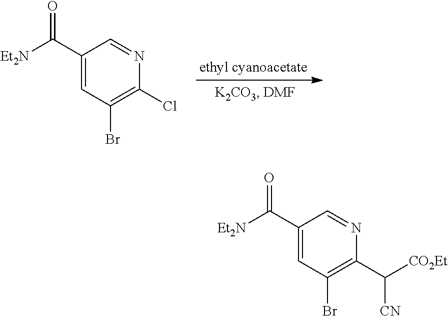

To a solution of 5-bromo-6-chloro-N,N-diethylnicotinamide (14.206 g, 48.722 mmol, 1.0 equiv) in DMF (81.2 mL) was added K₂CO₃ (53.869 g, 389.779 mmol, 8.0 equiv) followed by ethyl cyanoacetate (20.74 mL, 134.890 mmol, 4.0 equiv). The mixture was heated to 130° C. with stirring for 1 h and subsequently cooled to ambient temperature then 0° C. A solution of 4M HCl_{(aq.)} (200 mL) was added dropwise over 1 h with vigorous stirring. Next, H₂O (200 mL) was added and the mixture was stirred at 0° C. for 1 h and then filtered. The filter cake was washed with ice-cold H₂O (3×50 mL), and dried in vacuo to afford ethyl 2-(3-bromo-5-(diethylcarbamoyl)pyridin-2-yl)-2-cyanoacetate (15.409 g, 86%) as a yellow solid comprised of a 2:1 tautomeric mixture (determined via ¹H NMR analysis).

¹H NMR (400 MHz, CDCl₃) δ=15.32* (br, 0.5H), 8.55^{†} (d, J=1.76 Hz, 1H), 7.96^{†} (d, J=1.76 Hz, 1H), 7.87* (d, J=1.76 Hz, 0.5H), 7.84-7.79* (M, 0.5H), 5.41^{†} (s, 1H), 4.34-4.23 (M, 3H), 3.58-3.21 (m, 6H), 1.36-1.12 (m, 13.5H). ¹³C NMR (100 MHz, CDCl₃) δ=172.1, 166.0, 164.7, 163.0, 152.8, 149.8, 145.8, 143.8, 139.6, 134.8, 133.3, 122.3, 121.1, 118.0, 113.8, 112.7, 65.9, 63.9, 61.5, 45.7, 43.6, 39.9, 14.5, 14.4, 14.0, 12.8 ppm. LRMS (ES⁺) m/z [M+H]⁺ calcd for C₁₅H₁₉BrN₃O₃⁺ 368.06; Found 368.25. ^{†} Denotes ¹H NMR signal arising exclusively from the major tautomer; *denotes ¹H NMR signal arising exclusively from the minor tautomer; undesignated signals arise from both.

### 3-bromo-5-(diethylcarbamoyl)picolinic acid

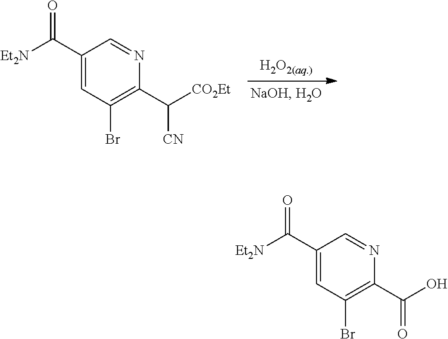

To a solution of NaOH (3.824 g, 95.516 mmol, 2.5 equiv.) in H₂O (64.3 mL) was added ethyl 2-(3-bromo-5-(diethylcarbamoyl)pyridin-2-yl)-2-cyanoacetate (14.216 g, 38.606 mmol, 1.0 equiv) with vigorous stirring. The resulting solution was heated to 40° C. and H₂O_{2(aq.)} (30%) (21.886 g, 193.032 mmol, 5.0 equiv) was carefully added dropwise over 1 h before heating to 90° C. for 1 h. The solution was cooled to ambient temperature, and then 4M HCl_{(aq.)} (29 mL) was added dropwise over 1 h with vigorous stirring. The resulting suspension was cooled at 0° C. for 1 h and filtered. The filter cake was washed with ice-cold H₂O (2×40 mL), and the solid collected. The combined aqueous filtrates were extracted with EtOAc (5×100 mL), dried over Na₂SO₄, and concentrated under reduced pressure. To the residue was added 8 mL H₂O, and the resulting mixture was cooled to 0° C. with stirring and the resulting precipitate was filtered then washed with ice-cold H₂O (2×4 mL) to afford a second crop. The combined solids were dried in vacuo to afford 3-bromo-5-(diethylcarbamoyl)picolinic acid (9.419 g, 81%) as a white solid. [0085]¹H NMR (400 MHz, DMSO-d₆) δ=13.97 (br s, 1H), 8.60 (d, J=1.68 Hz, 1H), 8.26 (d, J=1.68 Hz, 1H), 3.45 (q, J=6.92 Hz, 2H), 3.45 (q, J=6.84 Hz, 2H), 1.15 (t, J=6.78 Hz, 3H), 1.06 (t, J=6.74 Hz, 3H) ppm. ¹³C NMR (100 MHz, DMSO-d₆) δ=166.4, 165.3, 151.6, 145.0, 138.8, 135.4, 116.2, 43.0, 39.0, 13.9, 12.7 ppm. LRMS (ES⁺) m/z [M+H]⁺ calcd for C₁₁H₁₄BrN₂O₃⁺ 301.02; Found 301.29.

### 5-bromo-N,N-diethyl-6-(hydroxymethyl)nicotinamide

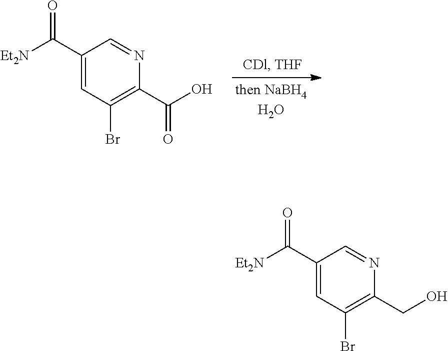

To a 0° C. cooled solution of 3-bromo-5-(diethylcarbamoyl)picolinic acid (8.101 g, 26.901 mmol, 1.0 equiv) in THF (134.5 mL) was added carbonyldiimidazole (4.798 g, 29.591 mmol, 1.1 equiv), and subsequently warmed to ambient temperature and stirred for 2.5 h. The solution was then added slowly via cannula to a 0° C. cooled solution of NaBH₄ (3.053 g, 80.703 mmol, 3.0 equiv) in H₂O (134.5 mL) with vigorous stirring, before warming to ambient temperature and stirring for 20 min. The solution was cooled to 0° C. and quenched by the addition of 1M HCl (110 mL) dropwise over 30 min. Upon warming to room temperature, EtOAc (250 mL) and brine (400 mL) were added, and the layers separated. The aqueous layer was further extracted with EtOAc (4×120 mL). The organic extracts were combined, washed with saturated NaHCO₃(aq.) (100 ML) then brine (100 mL), dried over Na₂SO₄, and concentrated under reduced pressure. The residue was taken up in 1:1 EtOAc/hexanes (100 mL), filtered over celite, and washed with additional solvent (200 mL). The filtrate was concentrated under reduced pressure and dried in vacuo to afford 5-bromo-N,N-diethyl-6-(hydroxymethyl)nicotinamide (7.406 g, 96%) as a pale-yellow oil.

¹H NMR (400 MHz, CDCl₃) δ=8.52 (d, J=1.7 Hz, 1H), 7.89 (d, J=1.7 Hz, 1H), 4.75 (d, J=4.7 Hz, 2H), 4.25 (br s, 1H), 3.65-3.41 (m, 2H), 3.36-3.15 (m, 2H), 1.32-1.08 (m, 6H) ppm. ¹³C NMR (100 MHz, CDCl₃) δ=166.8, 157.6, 144.1, 138.6, 133.2, 118.7, 63.4, 43.6, 39.9, 14.5, 12.9 ppm. LRMS (ES⁺) m/z [M+H]⁺ calcd for C₁₁H₁₆BrN₂O₂⁺ 287.04; Found 287.12.

### Example 2: Preparation of 5-bromo-N,N-diethyl-6-(hydroxymethyl)nicotinamide via N-Oxide

### 3-bromo-5-(diethylcarbamoyl)pyridine 1-oxide (3)

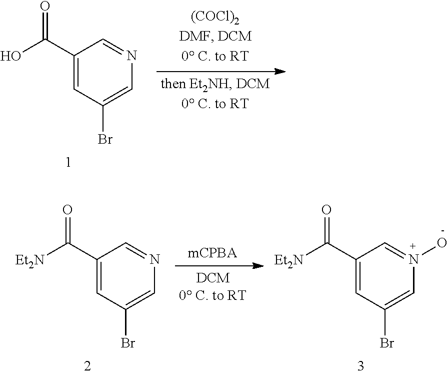

To a 0° C. cooled mixture of 5-bromonicotinic acid (10.000 g, 49.503 mmol, 1.0 equiv) in DCM (250 mL) was added oxalyl chloride (6.37 mL, 74.2 mmol, 1.5 equiv) slowly. To the suspension was added DMF (0.5 mL) dropwise, and the mixture was warmed to ambient temperature and stirred for 1 h. The mixture was cooled to 0° C. and a solution of diethylamine (25.61 mL, 247.5 mmol, 5.0 equiv) in DCM (250 mL) was added slowly via cannula. The mixture was warmed to ambient temperature and stirred for 30 min. H₂O (500 mL) was added, followed by 2M HCl (40 mL) until the pH=1 to 2. The layers were separated, and the aqueous layer was further extracted with DCM (3×200 mL). The organic extracts were combined and sequentially washed with saturated aqueous NaHCO₃ (1×250 mL) and brine (1×250 mL). The organic extract was dried over Na₂SO₄, and concentrated under reduced pressure.

To a 0° C. cooled solution of the resulting brown oil in DCM (200 mL) was added MCPBA (70-75% balance) (22.781 g, 99.006 mmol, 2.0 equiv). The mixture was warmed to ambient temperature and stirred for 18 h. To the solution was added saturated aqueous NaHCO₃ (500 mL) and then 1M NaOH (500 mL). The layers were separated, and the aqueous layer was further extracted with 10% IPA in DCM (3×200 mL). The organic layers were combined, dried over Na₂SO₄, filtered, and concentrated under reduced pressure. The residue was purified via chromatography on silica gel (EtOAc then 12% MeOH in EtOAc) and concentrated under reduced pressure. The resulting pale yellow oil was dissolved in DCM (50 mL), and to the solution was added hexanes (500 mL) slowly with vigorous stirring. The suspension was cooled to 0° C., filtered, and washed with 100 mL cold hexanes to afford 3 (11.041 g, 82%) as a white solid.

¹H NMR (400 MHz, CDCl₃) δ=8.32 (t, J=1.5 Hz, 1H), 8.09 (t, J=1.3 Hz, 1H), 7.35 (t, J=1.3 Hz, 1H), 3.54-3.46 (m, 2H), 3.29-3.21 (m, 2H), 1.24-1.12 (m, 6H) ppm. ¹³C NMR (100 MHz, CDCl₃) δ=164.3, 141.0, 136.3, 135.9, 126.4, 120.7, 43.5, 39.9, 14.4, 12.8 ppm. LRMS (ES⁺) m/z [M+H]⁺ calcd for C₁₀H₁₄BrN₂O₂⁺ 273.02; Found 273.12. IR (diamond, ATR) ν 3445, 3068, 2973, 2934, 1633 cm⁻¹.

### 5-bromo-6-chloro-N,N-diethylnicotinamide (4)

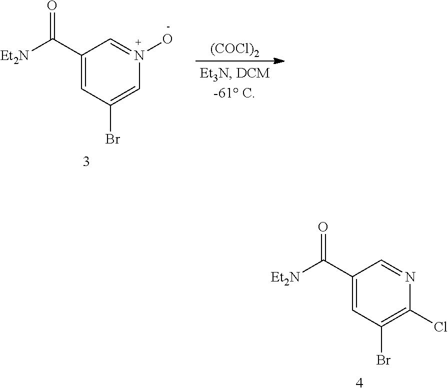

To a −61° C. cooled (CHCl₃/dry ice) solution of 3 (9.900 g, 36.395 mmol, 1.0 equiv) and Et₃N (10.15 mL, 72.79 mmol, 2.0 equiv) in DCM (180 mL) was added oxalyl chloride (6.24 mL, 72.8 mmol, 2.0 equiv) slowly dropwise. The mixture was stirred for 30 minutes. then MeOH (5 mL) was added slowly before warming to ambient temperature, then saturated aqueous NaHCO₃ (25 mL) was added. The solution was poured into 1M NaOH (600 mL) and the layers were separated. The aqueous layer was further extracted with DCM (3×150 mL). The organic extracts were combined, washed with brine (250 mL), dried over Na₂SO₄, and concentrated under reduced pressure. The residue was purified by chromatography on silica gel (25% EtOAc in hexanes) to afford 4 (9.442 g, 89%) as a crystalline white solid.

¹H NMR (400 MHz, CDCl₃) δ=8.34 (s, 1H), 7.96 (s, 1H), 3.59-3.42 (m, 2H), 3.36-3.18 (m, 2H), 1.28-1.10 (m, 6H) ppm. ¹³C NMR (100 MHz, CDCl₃) δ=166.0, 151.5, 145.3, 140.7, 133.0, 120.5, 43.6, 39.9, 14.4, 12.9 ppm. LRMS (ES⁺) m/z [M+H]* calcd for C₁₀H₁₃BrClN₂O⁺ 290.99; Found 291.00. IR (diamond, ATR) ν 2974, 2935, 1627, 1574 cm⁻¹.

### diethyl 2-(3-bromo-5-(diethylcarbamoyl)pyridin-2-yl)malonate (6)

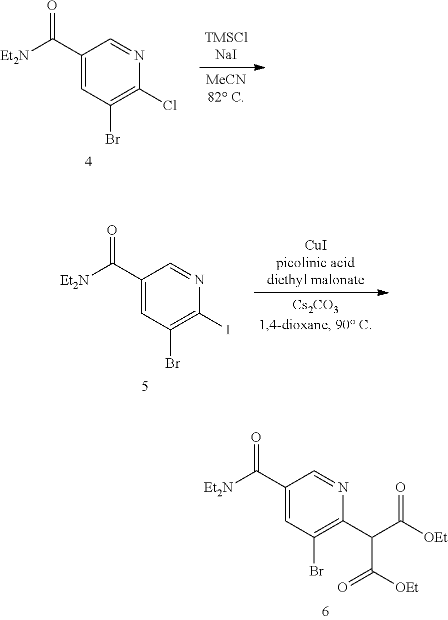

To a vigorously stirred mixture of 4 (5.000 g, 17.243 mmol, 1.0 equiv) and NaI (20.676 g, 137.94 mmol, 8.0 equiv) in acetonitrile (40 mL) was added TMSCl (3.28 mL, 25.86 mmol, 1.5 equiv) slowly. The mixture was stirred at ambient temperature for 30 min, then heated at reflux for 1 hour, with ¼ of the reaction volume removed and collected in a Dean-Stark receiver during this time period. The resulting yellow suspension was cooled to ambient temperature, diluted with DCM (150 mL), and added to a saturated aqueous NaHCO₃ solution (250 mL). With vigorous stirring, a saturated aqueous Na₂S₂O₃ solution (100 mL) was added, followed by 1M NaOH (80 mL). The resultant clear solution was transferred to a separatory funnel and the layers were separated. The aqueous layer was further extracted with DCM (3×100 mL). The organic extracts were combined, washed with brine (200 mL), dried over Na₂SO₄, and concentrated under reduced pressure.

The resulting pale orange solid was added to a sealable screw cap flask along with copper(I) iodide (0.164 g, 0.861 mmol, 0.05 equiv), picolinic acid (0.212 g, 1.72 mmol, 0.1 equiv), and CS₂CO₃ (16.854 g, 51.729 mmol, 3.0 equiv). 1,4-dioxane (43 mL) and diethyl malonate (5.26 mL, 34.5 mmol, 2.0 equiv) were added, and the flask capped. The mixture was stirred and heated at 90° C. for 16 h. The mixture was cooled to ambient temperature and filtered over celite, and the filter cake was washed with EtOAc (200 mL). The filtrate was added to H₂O (500 mL), then 1M HCl (10 mL) was added and the layers were separated. The aqueous layer was further extracted with EtOAc (2×200 mL). The organic extracts were combined, washed with brine (250 mL), dried over Na₂SO₄, and concentrated under reduced pressure. The residue was purified by chromatography on silica gel (20% EtOAc in hexanes to 50% EtOAc in hexanes) to afford 6 (5.461 g, 76%) as a pale yellow oil.

¹H NMR (400 MHz, CDCl₃) δ=8.52 (d, J=1.8 Hz, 1H), 7.92 (d, J=1.8 Hz, 1H), 5.22 (s, 1H), 4.34-4.23 (m, 4H), 3.62-3.44 (m, 2H), 3.38-3.19 (m, 2H), 1.32-1.10 (m, 12H) ppm. ¹³C NMR (100 MHz, CDCl₃) δ 166.62, 166.58, 152.5, 142.2, 138.9, 133.5, 121.9, 62.3, 59.9, 43.6, 39.8, 14.5, 14.1, 12.9 ppm. LRMS (ES⁺) m/z [M+H]⁺ calcd for C₁₇H₂₄BrN₂O₅⁺ 415.09; Found 415.19. IR (diamond, ATR) ν 2980, 2937, 1735, 1631 cm⁻¹.

### 3-bromo-5-(diethylcarbamoyl)-2-methylpyridine 1-oxide (8)

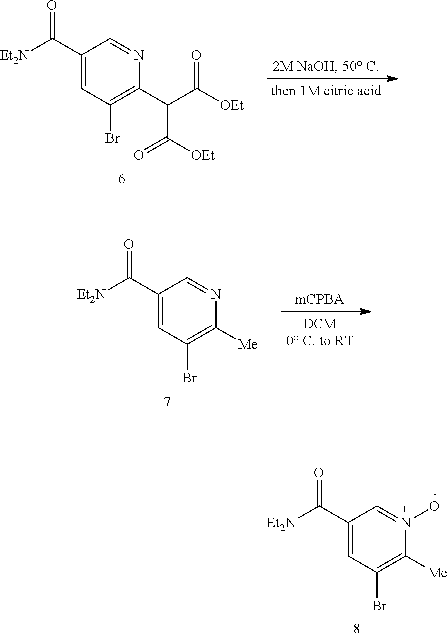

To a solution of 6 (5.350 g, 12.92 mmol, 1.0 equiv) in MeOH (130 mL) was added 2M aq. NaOH (32 mL), and the solution was stirred and heated at 50° C. for 16 h. To the resulting suspension, 1M aq. Citric acid (45 mL) was added to adjust the pH to 4, and the solution was stirred and heated at 60° C. for 24 h. The solution was cooled to ambient temperature and the MeOH was removed by concentration under reduced pressure. The solution was added to H₂O (250 mL) and extracted with DCM (3×200 mL). The organic layers were combined, washed with brine (250 mL), dried over Na₂SO₄, and concentrated under reduced pressure.

To a 0° C. cooled solution of the resulting residue in DCM (50 mL) was added MCPBA (70-75% balance) (5.946 g, 25.84 mmol, 2.0 equiv) slowly. The solution was warmed to ambient temperature and stirred for 22 h. The solution was added to 150 mL 1M NaOH and the layers were separated. The aqueous layer was further extracted with 10% isopropyl alcohol in DCM (3×100 mL). The organic extracts were combined, dried over Na₂SO₄, filtered, and concentrated under reduced pressure. The residue was purified by column chromatography on silica gel (EtOAc then 10% MeOH in EtOAc) to afford 8 (3.469 g, 94%) as a white solid.

¹H NMR (400 MHz, CDCl₃) δ=8.19 (d, J=0.9 Hz, 1H), 7.4 (d, J=0.9 Hz, 1H), 3.58-3.39 (m, 2H), 3.36-3.18 (m, 2H), 2.66 (s, 3H), 1.24-1.10 (m, 6H) ppm. ¹³C NMR (100 MHz, CDCl₃) δ=164.6, 150.2, 136.2, 133.0, 127.0, 122.1, 43.5, 39.9, 17.4, 14.4, 12.8 ppm. LRMS (ES⁺) m/z [M+H]⁺ calcd for C₁₁H₁₆BrN₂O₂⁺ 287.04; Found 287.12. IR (diamond, ATR) ν 3455, 2972, 2935, 1632 cm⁻¹.

### 5-bromo-N,N-diethyl-6-(hydroxymethyl)nicotinamide (9)

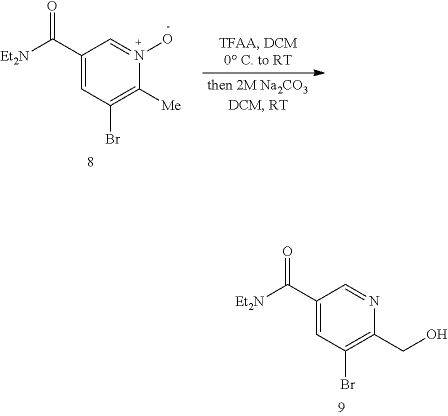

To a 0° C. cooled solution of 8 (1.301 g, 4.531 mmol, 1.0 equiv) in DCM (22.6 mL) was added trifluoroacetic anhydride (1.57 mL, 11.3 mmol, 2.5 equiv) dropwise. The solution was warmed to ambient temperature and stirred for 4 h before concentrating under reduced pressure. The residue was re-dissolved in DCM (22.6 mL) and 2M aq. Na₂CO₃ (45.2 mL) was added. The biphasic solution was stirred vigorously at ambient temperature for 18 h, then poured into H₂O (100 mL). The layers were separated and the aqueous layer was further extracted with DCM (3×50 mL). The organic extracts were combined, washed with brine (100 mL), dried over Na₂SO₄, and concentrated under reduced pressure. The residue was purified by column chromatography on silica gel (EtOAc) to afford 9 (1.119 g, 86%) as a yellow oil.

¹H NMR (400 MHz, CDCl₃) δ=8.53 (d, J=1.7 Hz, 1H), 7.90 (d, J=1.7 Hz, 1H), 4.77 (d, J=4.7 Hz, 2H), 4.23 (t, J=4.7 Hz, 1H), 3.64-3.46 (m, 2H), 3.38-3.18 (m, 2H), 1.32-1.08 (m, 6H) ppm. ¹³C NMR (100 MHz, CDCl₃) δ=166.8, 157.6, 144.1, 138.6, 133.2, 118.7, 63.4, 43.6, 39.9, 14.5, 12.9 ppm. LRMS (ES⁺) m/z [M+H]⁺ calcd for C₁₁H₁₆BrN₂O₂ 287.04; Found 287.12. IR (diamond, ATR) ν 3412, 2972, 2934, 1624, 1588 cm⁻¹.

### Example 3: Preparation of N,N-diethyl-8-methyl-7a,8,9,10-tetrahydro-7H-indolo[7,1-fg][1,7]naphthyridine-10-carboxamide (14, (±)-JRT)

Compound 14 is prepared according to the method described in PCT Application No. PCT/2022/024626.

### 5-bromo-N,N-diethyl-6-(hydroxymethyl)-1-methyl-1,2,3,6-tetrahydropyridine-3-carboxamide (10, major diastereomer)

### 5-bromo-N,N-diethyl-6-(hydroxymethyl)-1-methyl-1,2,3,6-tetrahydropyridine-3-carboxamide (11, minor diastereomer)

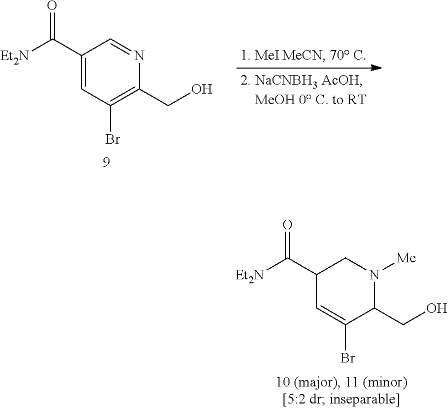

To a solution of 9 (0.980 g, 3.413 mmol, 1.0 equiv) in MeCN (4.25 mL) in a vial was added Mel (1.28 mL, 20.5 mmol, 6.0 equiv). The vial was capped and the solution was heated with stirring at 70° C. for 24 h then subsequently cooled to ambient temperature. To the mixture was added EtOAc (8.5 mL) followed by hexanes (8.5 mL) with vigorous stirring. The suspension was cooled to 0° C., filtered, and washed with hexanes (2×5 mL). The resulting yellow solid was dried under reduced pressure and used directly in the next step.

To a 0° C. cooled solution of the resulting methyl pyridinium salt (1.285 g, 2.995 mmol, 1.0 equiv) in MeOH (30 mL) was added AcOH (0.51 mL, 8.9 mmol, 3.0 equiv) followed by the dropwise addition of NaCNBH₃ (0.565 g, 8.98 mmol, 3.0 equiv) in MeOH (6 mL). The solution was warmed to ambient temperature and stirred for 16 h, then concentrated under reduced pressure. The residue was dissolved in EtOAc (100 mL) and added to 1M NaOH (200 mL). The layers were separated, and the aqueous layer was further extracted with EtOAc (3×100 mL). The organic extracts were combined and washed with brine (150 mL), dried over Na₂SO₄, and concentrated under reduced pressure. The residue was purified by column chromatography on silica gel (3% MeOH in DCM) to afford an inseparable mixture of diastereomers 10 (major diastereomer) and 11 (minor diastereomer) (0.726 g, 70%), 5:2 dr) as a pale yellow oil.

¹H NMR (400 MHz, CDCl₃) δ=6.21 (s, 1H), 6.11* (d, J=2.8 Hz, 0.4H), 3.94^{†} (dd, J=2.0 Hz, 1H), 3.88-3.78 (m, 1.4H), 3.70-3.64* (m, 0.4H), 3.59* (dd, J=8.8, 11.6 Hz, 0.4H), 3.54-3.47^{†} (m, 1H), 3.34 (quint, J=7.3 Hz, 5.6H), 3.21* (dd, J=9.4, 13.6 Hz, 0.4H), 3.14-3.08* (m, 0.4H), 3.02-2.92^{†} (m, 2H), 2.88-2.80^{†} (m, 1H), 2.76* (dd, J=5.1, 14 Hz, 0.4H), 2.55* (s, 1.2H), 2.45^{†} (s, 3H), 2.24-2.14 (m, 4.2H), 1.09 (t, J=7.1 Hz, 4.2H) ppm. ¹³C NMR (100 MHz, CDCl₃) δ=170.22, 170.15, 130.4, 127.6, 123.3, 122.9, 68.6, 67.6, 60.9, 59.6, 53.6, 47.0, 43.5, 42.9, 42.2, 42.1, 41.3, 40.7, 40.4, 36.7, 15.1, 14.9, 13.2, 13.1 ppm. LRMS (ES⁺) m/z [M+H]⁺ calcd for C₁₂H₂₂BrN₂O₂* 305.09; Found 305.14. IR (diamond, ATR) ν 3418, 2970, 2934, 2799, 1629 cm⁻¹. ^{†} Denotes ¹H NMR signal arising exclusively from the major diastereomer; *denotes ¹H NMR signal arising exclusively from the minor diastereomer; undesignated signals arise from a mixture of both.

### N,N-diethyl-6-(hydroxymethyl)-5-(1H-indol-7-yl)-1-methyl-1,2,3,6-tetrahydropyridine-3-carboxamide (12, major diastereomer)

### N,N-diethyl-6-(hydroxymethyl)-5-(1H-indol-7-yl)-1-methyl-1,2,3,6-tetrahydropyridine-3-carboxamide (13, minor diastereomer)

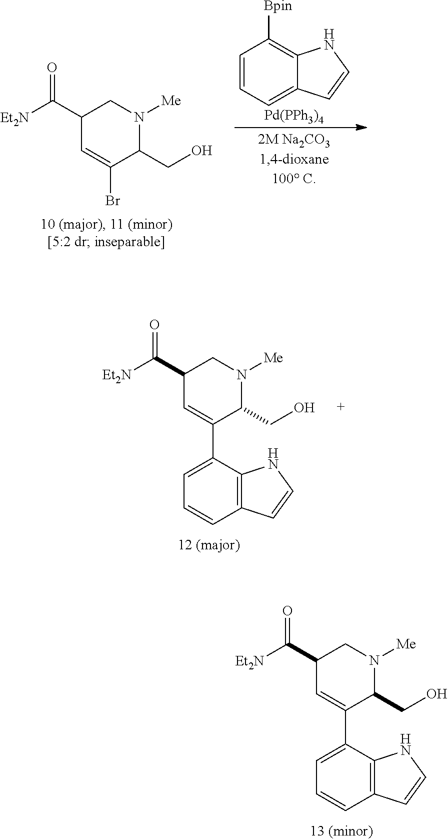

A mixture of diastereomers 10 and 11 (5:2 dr) (0.698 g, 2.29 mmol, 1.0 equiv), 1,4-dioxane (22.9 mL), indole-7-boronic acid pinacol ester (0.834 g, 3.43 mmol, 1.5 equiv), and 2M aq. Na₂CO₃ (2.29 mL) were added to a vial and the solution was sparged with N₂ for 10 min before the addition of Pd(PPh₃)₄ (0.132 g, 0.114 mmol, 0.05 equiv). The vial was capped and the mixture was heated with stirring at 100° C. in a preheated oil bath for 4 h. The mixture was cooled to ambient temperature, added to H₂O (400 mL), and extracted with EtOAc (3×150 mL). The organic extracts were combined, washed with brine (200 mL), dried over Na₂SO₄, and concentrated under reduced pressure. The residue was purified by column chromatography on silica gel (2% MeOH in DCM to 10% MeOH in DCM) to afford 12 (major diastereomer) (0.398 g, 51%) and 13 (minor diastereomer) (0.148 g, 19%) as off white semi-solids.

Major Diastereomer, 12. ¹H NMR (400 MHz, CDCl₃) δ=9.46 (s, 1H), 7.56 (d, J=7.8 Hz, 1H), 7.22 (t, J=2.8 Hz, 1H), 7.06 (t, J=7.3 Hz, 1H), 7.00 (dd, J=0.9, 8.4 Hz, 1H), 6.53 (dd, J=2.1, 3.2 Hz, 1H), 5.92 (s, 1H), 3.79 (dd, J=3.0, 11.2 Hz, 1H), 3.74-3.66 (m, 1H), 3.50-3.28 (m, 5H), 3.26-3.08 (m, 3H), 3.07-2.96 (m, 1H), 2.52 (s, 2H), 1.23 (t, J=7.1 Hz, 3H), 1.10 (t, J=7.1 Hz, 3H) ppm. ¹³C NMR (100 MHz, CDCl₃) δ=171.7, 137.3, 135.4, 128.11, 128.05, 124.9, 124.1, 121.4, 119.9, 119.4, 102.4, 66.8, 59.1, 54.0, 43.2, 42.0, 40.3, 39.3, 14.9, 13.1 ppm. LRMS (ES⁺) m/z [M+H]⁺ calcd for C₂₀H₂₈N₃O₂⁺ 342.22; Found 342.32. IR (diamond, ATR) ν 3267, 2970, 2932, 1615 cm⁻¹.

Minor Diastereomer, 13. ¹H NMR (400 MHz, CDCl₃) δ=9.86 (s, 1H), 7.55 (d, J=7.8 Hz, 1H), 7.29-7.24 (m, 1H), 7.07 (t, J=7.4 Hz, 1H), 6.98 (dd, J=0.6, 7.6 Hz, 1H), 6.51 (dd, J=2.1 Hz, 3.2 Hz, 1H), 6.08-6.04 (m, 1H0, 3.76-3.68 (m, 1H), 3.64-3.28 (m, 8H), 3.15-3.06 (m, 1H), 2.99 (dd, J=5.7, 13.2 Hz, 1H), 2.66 (s, 3H), 1.26 (t, J=7.2 Hz, 3H), 1.17 (t, J=7.1 Hz, 3H) ppm. ¹³C NMR (100 MHz, CDCl₃) δ=173.0, 135.7, 135.0, 128.4, 125.6, 125.4, 123.8, 120.0, 119.4, 119.0, 102.1, 64.8, 60.9, 48.6, 42.7, 42.3, 40.6, 34.8, 15.1, 13.3 ppm. LRMS (ES⁺) m/z [M+H]⁺ calcd for C₂₀H₂₈N₃O₂⁺342.22; Found 342.32. IR (diamond, ATR) ν 3270, 2973, 2934, 1613 cm⁻¹.

### N,N-diethyl-8-methyl-7a,8,9,10-tetrahydro-7H-indolo[7,1-g][1,7]naphthyridine-10-carboxamide (14, (±)-JRT)

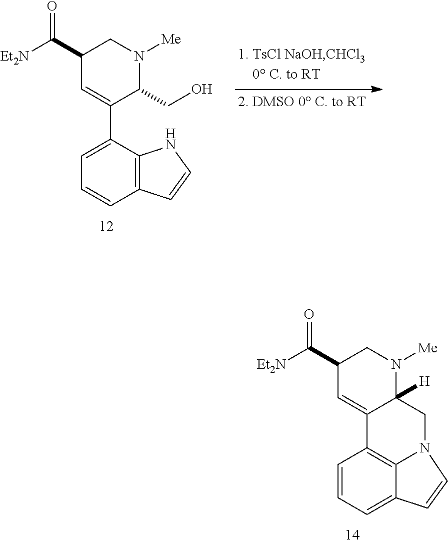

To a 0° C. cooled solution of 12 (0.250 g, 0.732 mmol, 1.0 equiv) in CHCl₃ (7.3 mL) was added freshly crushed NaOH (0.234 g, 5.86 mmol, 8.0 equiv). A solution of TsCl (0.167 g, 0.878 mmol, 1.2 equiv) in CHCl₃ (1.5 mL) was added dropwise over 10 minutes. The mixture was warmed to ambient temperature and stirred for 1.5 h. The mixture was cooled to 0° C., and DMSO (3.7 mL) was added slowly before warming to ambient temperature and stirring for 1 h. The mixture was partitioned in H₂O (250 mL) and EtOAc (200 mL) and the layers were separated. The aqueous layer was further extracted with EtOAc (3×100 mL). The organic extracts were combined, washed with brine (250 mL), dried over Na₂SO₄, and concentrated under reduced pressure. The residue was purified by column chromatography on silica gel (8% MeOH in EtOAc to 12% MeOH in EtOAc) to afford 14 (0.168 g, 71%) as an off white semi-solid.

¹H NMR (400 MHz, CDCl₃) δ=7.5 (d, J=7.9 Hz, 1H), 7.31 (d, J=7.3 Hz, 1H), 7.08-7.04 (m, 2H), 6.46 (d, J=3.0 Hz, 1H), 6.31 (s, 1H), 4.66 (dd, J=5.4, 11.2 Hz, 1H), 3.90-3.82 (m, 1H), 3.80 (t, J=11.1 Hz, 1H), 3.54-3.40 (m, 5H), 3.05 (dd, J=5.0, 11.2 Hz, 1H), 2.95 (t, J=10.7 Hz, 1H), 2.59 (s, 3H), 1.26 (t, J=7.1 Hz, 3H), 1.18 (t, J=7.1 Hz, 3H) ppm. ¹³C NMR (100 MHz, CDC) δ=171.2, 133.2, 132.5, 126.3, 126.2, 120.3, 120.0, 118.91, 118.88, 114.9, 101.3, 60.5, 55.8, 48.0, 44.0, 42.1, 40.3, 39.9, 15.0, 13.2 ppm. LRMS (ES⁺) m/z [M+H]⁺ calcd for C₂₀H₂₆N₃O⁺ 324.21; Found 324.29. IR (diamond, ATR) ν 2972, 2869, 2798, 1636 cm⁻¹.

### Example 4: Preparation of N,N-diethyl-8-methyl-7a,8,9,10-tetrahydro-7H-indolo[7,1-fg][1,7]naphthylpyridine-10-carboxamide (15)

Compound 15 is prepared according to the method described in PCT Application No. PCT/2022/024626.

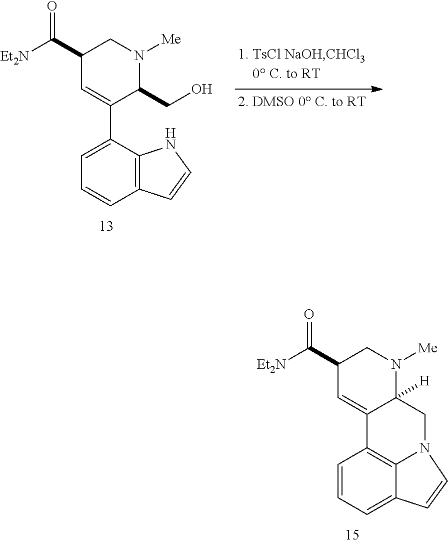

To a 0° C. cooled solution of 13 (0.130 g, 0.381 mmol, 1.0 equiv) in CHCl₃ (3.8 mL) was added freshly crushed NaOH (0.122 g, 3.05 mmol, 8.0 equiv). A solution of TsCl (0.087 g, 0.46 mmol, 1.2 equiv) in CHCl₃ (0.76 mL) was added dropwise over 10 minutes. The mixture was warmed to ambient temperature and stirred for 1.5 h. The mixture was cooled to 0° C., and DMSO (1.9 mL) was added slowly before warming to ambient temperature and stirring for 3 h. The mixture was partitioned in H₂O (200 mL) and EtOAc (150 mL) and the layers were separated. The aqueous layer was further extracted with EtOAc (3×50 mL). The organic extracts were combined, washed with brine (100 mL), dried over Na₂SO₄, and concentrated under reduced pressure. The residue was purified by column chromatography on silica gel 8% MeOH in EtOAc to 12% MeOH in EtOAc) to afford 15 (0.044 g, 36%) as a brown semi-solid.

¹H NMR (400 MHz, CDCl₃) δ=7.48 (d, J=7.9 Hz, 1H), 7.22 (d, J=7.2 Hz, 1H), 7.08-7.02 (m, 2H), 6.45 (d, J=3.0 Hz, 1H), 6.37 (dd, J=2.0, 3.6 Hz, 1H), 4.50 (dd, J=5.5, 11.2 Hz, 1H), 4.02 (t, J=11.2 Hz, 1H), 3.66-3.60 (m, 1H), 3.56-3.30 (m, 5H), 3.15 (dd, J=5.7, 12.2 Hz, 1H), 2.83 (dd, J=4.8, 12.2 Hz, 1H), 2.62 (s, 3H), 1.27 (t, J=7.0 Hz, 3H), 1.13 (t, J=7.0 Hz, 3H) ppm. ¹³C NMR (100 MHz, CDCl) δ=171.5, 133.4, 133.4, 126.5, 126.2, 120.4, 120.1, 119.9, 118.9, 114.2, 101.2, 58.6, 52.5, 47.9, 43.6, 42.0, 40.3, 37.4, 15.0, 13.2 ppm. LRMS (ES⁺) m/z [M+H]⁺ calcd for C₂₀H₂₆N₃O⁺ 324.21; Found 324.29. IR (diamond, ATR) ν 2969, 2932, 2871, 2791, 1634 cm⁻¹.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of preparing a compound of Formula I: 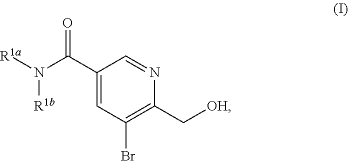
the method comprising:
(a1) forming a first reaction mixture comprising a first activating agent, and a compound of Formula II: 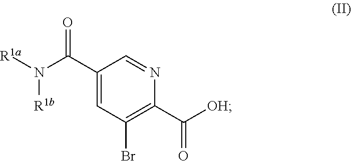
and
(a2) adding the first reaction mixture to a solution comprising a reducing agent, under conditions suitable to prepare the compound of Formula I,
wherein
R^{1a} and R^{1b} are each independently C₁₋₆ alkyl, C₁₋₆ alkoxy, C₁₋₆ alkoxyalkyl, C₁₋₆ haloalkyl, C₁₋₆ haloalkoxy, or C₃₋₆ cycloalkyl;
alternatively, R^{1a} and R^{1b} are combined to form a 4 to 8 membered heterocycloalkyl having 1 to 2 heteroatoms, each independently N, O, or S.

2. The method of claim 1, wherein R^{1a} and R^{1b} are each independently C₁₋₆ alkyl.

3. The method of claim 1 or 2, wherein R^{1a} and R^{1b} are each independently methyl, ethyl, n-propyl, or i-propyl.

4. The method of any one of claims 1 to 3, wherein
the compound of Formula I is a compound of Formula Ia: 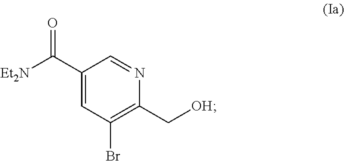
the compound of Formula II is a compound of Formula IIa:

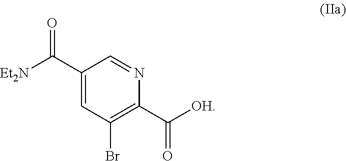

5. The method of any one of claims 1 to 4, wherein the first activating agent comprises carbonyldiimidazole, dicyclohexylcarbodiimide (DCC), N,N′-diisopropylcarbodiimide (DIC), 1-ethyl-3-(3-dimethylaminopropyl)carbodiimide (EDC), 1-ethyl-3-(3-dimethylaminopropyl)carbodiimide HCl (EDAC), bis[[4-(2,2-dimethyl-1,3-dioxolyl)]methyl]carbodiimide (BDDC), N-cyclohexyl-N′-(2-morpholinoethyl)carbodiimide methyl-p-toluenesulfonate, 1-[3-(dimethylamino)propyl]-3-ethylcarbodiimide methiodide, thionyl chloride, oxalyl chloride, 2-ethoxy-1-ethoxycarbonyl-1,2-dihydroquinoline (EEDQ), carbonyl diimidazole, bis(1,2,4-triazolyl)methanone, n-propanephosphonic acid anhydride, ethylmethylphosphonic anhydide (EMPA), cyanuric chloride, 2-chloro-4,6-dimethoxy-1,3,5-triazine (CDMT), 4-(4,6-dimethoxy-1,3,5-triazin-2-yl)-4-methylmorphonium chloride (DMTMM), guanidinium salt, uronium salt, hexafluorophosphate benzotriazole tetramethyl uronium (HBTU), hexafluorophosphate azabenzotriazole tetramethyl uronium (HATU), 2-(1H-Benzotriazole-1-yl)-1,1,3,3-tetramethylaminium tetrafluoroborate (TBTU), O-(2-Oxo-(2H)pyridyl)-N,N,N′,N′-tetramethyluronium tetrafluoroborate (TPTU), or N-[[[(1-Cyano-2-ethoxy-2-oxoethylidene)amino]oxy](dimethylamino)methylene]-N-methyl-methanaminium tetrafluoroborate (TOTU).

6. The method of any one of claims 1 to 5, wherein the first activating agent comprises carbonyldiimidazole.

7. The method of any one of claims 1 to 6, wherein the reducing agent comprises lithium borohydride (LiBH₄), sodium borohydride (NaBH₄), calcium borohydride (Ca(BH₄)₂), sodium cyanoborohydride (NaBH₃CN), sodium triacetoxyborohydride (NaBH(OAc)₃), diisobutylaluminum hydride (DIBAL), or lithium aluminum hydride (LiAlH₄).

8. The method of any one of claims 1 to 7, wherein the reducing agent comprises sodium borohydride.

9. The method of any one of claims 1 to 8, wherein
the first activating agent is present in an amount of 1 to 5 molar equivalents to the compound of Formula II; and
the reducing agent is present in an amount of 1 to 5 molar equivalents to the compound of Formula II.

10. The method of any one of claims 1 to 9, wherein
the first activating agent is present in an amount of 1 to 3 molar equivalents to the compound of Formula II; and
the reducing agent is present in an amount of 2 to 4 molar equivalents to the compound of Formula II.

11. The method of any one of claims 1 to 10, wherein the compound of Formula II is prepared by the method comprising:
(a1) forming the first reaction mixture comprising carbonyldiimidazole and the compound of Formula II; and
(a2) adding the first reaction mixture to the solution comprising sodium borohydride, under conditions suitable to prepare the compound of Formula I.

12. The method of any one of claims 1 to 11, wherein the compound of Formula II is prepared by the method comprising:
(b1) forming a second reaction mixture comprising a first inorganic base, water, and a compound of Formula IIIa: 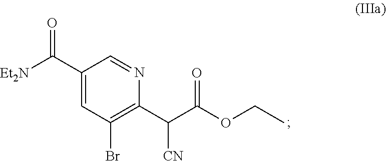
and
(b2) adding an oxidizing agent to the second reaction mixture, under conditions suitable to prepare the compound of Formula II.

13. The method of claim 12, wherein the first inorganic base comprises lithium hydroxide, sodium hydroxide, potassium hydroxide, sodium carbonate, potassium carbonate, cesium carbonate, or sodium acetate.

14. The method of claim 12 or 13, wherein the first inorganic base comprises sodium hydroxide.

15. The method of any one of claims 12 to 14, wherein the oxidizing agent comprises hydrogen peroxide, tert-butylhydroperoxide, meta-chloroperoxybenzoic acid (mCPBA), peracetic acid, or oxone.

16. The method of any one of claims 12 to 15, wherein the oxidizing agent comprises hydrogen peroxide.

17. The method of any one of claims 12 to 16, wherein
the first inorganic base is present in an amount of 1.5 to 3.5 molar equivalents to the compound of Formula IIIa; and
the oxidizing agent is present in an amount of 4 to 8 molar equivalents to the compound of Formula IIIa.

18. The method of any one of claims 12 to 17, wherein the compound of Formula IIIa is prepared by the method comprising:
(b1) forming the second reaction mixture comprising sodium hydroxide, water, and the compound of Formula IIIa: 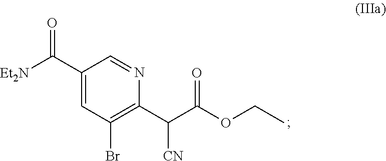
and
(b2) adding hydrogen peroxide to the second reaction mixture, under conditions suitable to prepare the compound of Formula II.

19. The method of any one of claims 12 to 18, wherein the compound of Formula III is prepared by the method comprising:
(c) forming a third reaction mixture comprising ethylcyanoacetate, a second inorganic base, and a compound of Formula IVa: 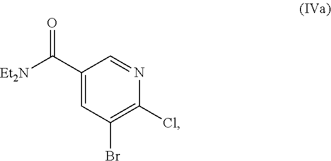
under conditions suitable to prepare the compound of Formula IIIa.

20. The method of claim 19, wherein the second inorganic base comprises sodium carbonate, potassium carbonate, cesium carbonate, sodium hydride, lithium hexamethyldisiloxane (LiHMDS), potassium hexamethyldisiloxane (KHMDS), sodium hexamethyldisiloxane (NaHMDS), or lithium diisopropyl amine (LDA).

21. The method of claim 19 or 20, wherein the second inorganic base comprises potassium carbonate.

22. The method of any one of claims 19 to 21, wherein the third reaction mixture further comprises dimethylformamide.

23. The method of any one of claims 19 to 22, wherein
the ethylcyanoacetate is present in an amount of 2.0 to 5.0 molar equivalents to the compound of Formula IVa; and
the second inorganic base is present in an amount of 4 to 10 molar equivalents to the compound of Formula IVa.

24. The method of any one of claims 19 to 23, wherein the compound of Formula IVa is prepared by the method comprising:
(c) forming the third reaction mixture comprising ethylcyanoacetate, potassium carbonate, dimethylformamide, and the compound of Formula IVa, under conditions suitable to prepare the compound of Formula IIIa.

25. The method of any one of claims 19 to 24, wherein the compound of Formula IVa is prepared by the method comprising:
(d1) forming a fourth reaction mixture comprising a second activating agent, and a compound of Formula V: 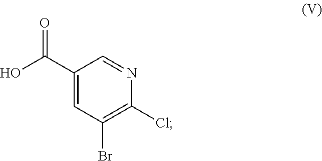 [SMILES: O=C(O)c1cnc(Cl)c(Br)c1]
and
(d2) adding diethylamine to the fourth reaction mixture, under conditions suitable to prepare the compound of Formula IVa.

26. The method of claim 25, wherein the second activating agent comprises oxalyl chloride, thionyl chloride, phosphorous oxychloride, (chloromethylene)dimethyliminium chloride, phosphorous(V) chloride, phosphorous(III) chloride, carbonyldiimidazole, dicyclohexylcarbodiimide (DCC), N,N′-diisopropylcarbodiimide (DIC), 1-ethyl-3-(3-dimethylaminopropyl)carbodiimide (EDC), 1-ethyl-3-(3-dimethylaminopropyl)carbodiimide HCl (EDAC), bis[[4-(2,2-dimethyl-1,3-dioxolyl)]methyl]carbodiimide (BDDC), N-cyclohexyl-N′-(2-morpholinoethyl)carbodiimide methyl-p-toluenesulfonate, 1-[3-(dimethylamino)propyl]-3-ethylcarbodiimide methiodide, thionyl chloride, oxalyl chloride, 2-ethoxy-1-ethoxycarbonyl-1,2-dihvdroquinoline (EEDQ), carbonyl diimidazole, bis(1,2,4-triazolyl)methanone, n-propanephosphonic acid anhydride, ethylmethylphosphonic anhydride (EMPA), cyanuric chloride, 2-chloro-4,6-dimethoxy-1,3,5-triazine (CDMT), 4-(4,6-dimethoxy-1,3,5-triazin-2-yl)-4-methylmorphonium chloride (DMTMM), guanidinium salt, uronium salt, hexafluorophosphate benzotriazole tetramethyl uronium (HBTU), hexafluorophosphate azabenzotriazole tetramethyl uronium (HATU), 2-(1H-Benzotriazole-1-yl)-1,1,3,3-tetramethylaminium tetrafluoroborate (TBTU), O-(2-Oxo-1(2H)pyridyl)-N,N,N′,N′-tetramethyluronium tetrafluoroborate (TPTU), or N-[[[(1-Cyano-2-ethoxy-2-oxoethylidene)amino]oxy](dimethylamino)methylene]-N-methyl-methanaminium tetrafluoroborate (TOTU).

27. The method of claim 25 or 26, wherein the second activating agent comprises oxalyl chloride.

28. The method of any one of claims 25 to 27, wherein
the second activating agent is present in an amount of 1 to 3 molar equivalents to the compound of Formula V; and
the diethylamine is present in an amount of 3 to 6 molar equivalents to the compound of Formula V.

29. The method of any one of claims 25 to 28, wherein
(d1) forming the fourth reaction mixture comprising oxalyl chloride, and the compound of Formula V; and
(d2) adding diethylamine to the fourth reaction mixture, under conditions suitable to prepare the compound of Formula IVa.

30. The method of any one of claims 25 to 29, comprising:
(d1) forming the fourth reaction mixture comprising oxalyl chloride, and the compound of Formula V: 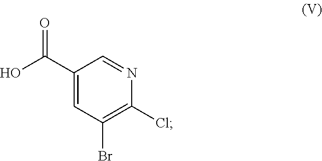 [SMILES: O=C(O)c1cnc(Cl)c(Br)c1]
(d2) adding diethylamine to the fourth reaction mixture, under conditions suitable to prepare the compound of Formula IVa: 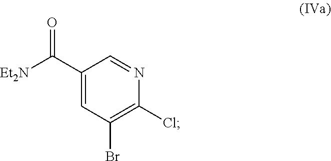
(c) forming the third reaction mixture comprising ethylcyanoacetate, potassium carbonate, dimethylformamide, and the compound of Formula IVa, under conditions suitable to prepare the compound of Formula IIIa: 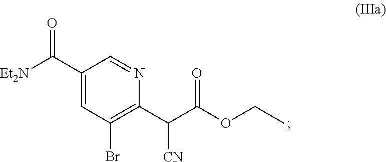
(b1) forming the second reaction mixture comprising sodium hydroxide, water, and the compound of Formula IIIa;
(b2) adding hydrogen peroxide to the second reaction mixture, under conditions suitable to prepare the compound of Formula Ha having the structure: 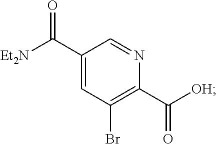
(a1) forming the first reaction mixture comprising carbonyldiimidazole and the compound of Formula IIa; and
(a2) adding the first reaction mixture to the solution comprising sodium borohydride, under conditions suitable to prepare the compound of Formula I having the structure:

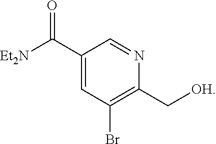

31. A method of preparing a compound of Formula IX: 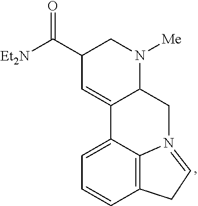
the method comprising:
(d1) forming a fourth reaction mixture comprising oxalyl chloride, and a compound of Formula V: 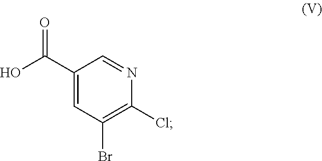 [SMILES: O=C(O)c1cnc(Cl)c(Br)c1]
(d2) adding diethylamine to the fourth reaction mixture, under conditions suitable to prepare a compound of Formula IVa: 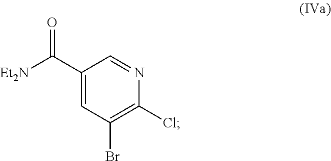
(c) forming a third reaction mixture comprising ethylcyanoacetate, potassium carbonate, dimethylformamide, and the compound of Formula IVa, under conditions suitable to prepare a compound of Formula IIIa: 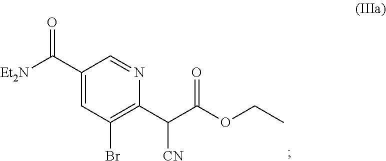
(b1) forming a second reaction mixture comprising sodium hydroxide, water, and the compound of Formula IIIa;
(b2) adding hydrogen peroxide to the second reaction mixture, under conditions suitable to prepare a compound of Formula IIa: 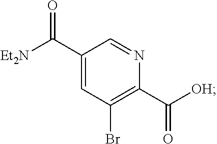
(a1) forming a first reaction mixture comprising carbonyldiimidazole and the compound of Formula Ia;
(a2) adding the first reaction mixture to a solution comprising sodium borohydride, under conditions suitable to prepare a compound of Formula Ia: 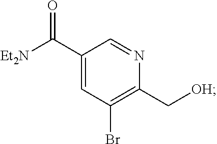
(e1) forming a fifth reaction mixture comprising methyliodide, and the compound of Formula Ia, under conditions suitable to prepare a methyl pyridinium salt of Formula VI: 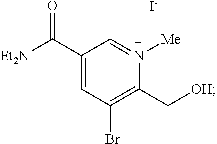
(e2) forming a sixth reaction mixture comprising sodium cyanoborohydride, and the methyl pyridinium salt of Formula VI, under conditions suitable to prepare a compound of Formula VII: 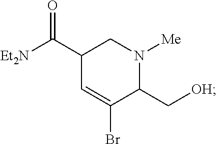
(f) forming a seventh reaction mixture comprising indole-7-boronic acid pinacol ester, sodium carbonate, Pd(PPh₃)₄, and the compound of Formula VI, under conditions suitable to prepare a compound of Formula VIII: 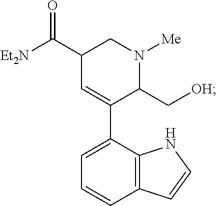
and
(g) forming an eighth reaction mixture comprising tosyl-chloride, sodium hydroxide, and the compound of Formula VIII, under conditions suitable to prepare the compound of Formula IX.
